(19) United States
(12) Patent Application Publication
(10) Pub. No.: US 2014/0340640 A1
(43) Pub. Date: Nov. 20, 2014

(54) PARKINSON'S DISEASE SCREENING USING THE KING-DEVICK TEST

(71) Applicant: King-Devick Test LLC, Oakbrook Terrace, IL (US)

(72) Inventor: Steven Devick, Downers Grove, IL (US)

(21) Appl. No.: 14/451,054

(22) Filed: Aug. 4, 2014

Related U.S. Application Data
(63) Continuation of application No. 13/355,000, filed on Jan. 20, 2012, now abandoned.

(51) Int. Cl. A61B 5/00; A61B 3/113

(52) U.S. Cl. CPC A61B 5/4082; A61B 3/113
USPC 351/209; 351/246

(57) ABSTRACT

A method of screening for Parkinson's disease, may include administering to a person in need of such screening a King-Devick Test, determining whether the person has a “normal” or “abnormal” result for the King-Devick Test, and recommending the person undergo additional testing and/or treatment for Parkinson's disease if the result is “abnormal.”

### CROSS-REFERENCE TO RELATED APPLICATIONS

This application claims priority to co-pending U.S. patent application Ser. No. 13/355,000, filed Jan. 20, 2012.

### BACKGROUND

Parkinson's disease (PD) is a progressive, neurodegenerative disorder of unknown origin primarily characterized by degeneration of the dopamine-producing neurons in the substantia nigra region of the midbrain. PD onset is marked by a variety of motor and non-motor dysfunctions that vary from person to person; however, oculomotor changes are reported as being an early and consistent biomarker of PD.

### SUMMARY

The inventor realized that his King-Devick Test (the “K-D Test” or “Test”), widely recognized as a tool to evaluate saccadic eye movements, is ideally suited for use as a rapid, easy-to-administer screening tool to evaluate individuals having, suspected of having, or at risk for developing PD. Under normal conditions, the K-D Test is administered, and an objective conclusion is reached, in around one minute for adults and a few minutes for children. The K-D Test can therefore be used in a variety of situations in which rapid, easy to administer PD screening is beneficial, such as a during an examination by a medical professional or as part of a home- or self-examination program designed for people having, suspected of having, or at risk for developing PD, and the like.

### BRIEF DESCRIPTION OF THE DRAWINGS

FIG. 1 depicts a demonstration page of a King-Devick Test booklet.

### DETAILED DESCRIPTION

The King-Devick Test was developed in the early 1980's as an objective tool to evaluate saccadic eye movements. The Test is widely used by reading teachers in schools, medical professionals, or parents at home to determine whether a student's poor reading performance is related to deficiencies in their ability to move their eyes efficiently. Children that score below the expected norm are, almost always, not efficient readers.

Upon becoming acquainted with research suggesting the predictive value of oculomotor assessment, the inventor realized that the K-D Test, which he co-invented, could be used to provide a rapid screen for PD. For adults, the Test is normally completed in about one minute; six-year-old children complete the Test in about four minutes.

In one embodiment, the K-D Test is administered as part of general screening examinations to healthy patient populations in order to provide a rapid, objective, and easy-to-administer screen for PD or other causes of suboptimal nervous system function.

In another embodiment, the K-D Test is administered to persons having PD, suspected of having PD, or at risk for developing PD as a method for early detection of PD, or as a method to monitor progression of PD. Because oculomotor dysfunctions are an early biomarker of PD, the K-D Test is particularly useful as an objective method to screen for PD. In this scenario, the K-D Test is administered soon after the patient first notices any subtle PD symptoms, and the result informs the patient whether or not he or she should seek medical treatment for PD. Moreover, due to its high level of sensitivity, the K-D Test is also administered to patients at risk for developing PD (e.g., geriatric patients, and patients with a family history of PD) before the onset of any perceivable PD symptoms, regardless of how subtle, providing a very early method of detection. Such at-risk persons could self-administer the K-D Test (or have it administered to them) every day, regardless of whether they feel well, as an easy way to detect minute changes in PD status.

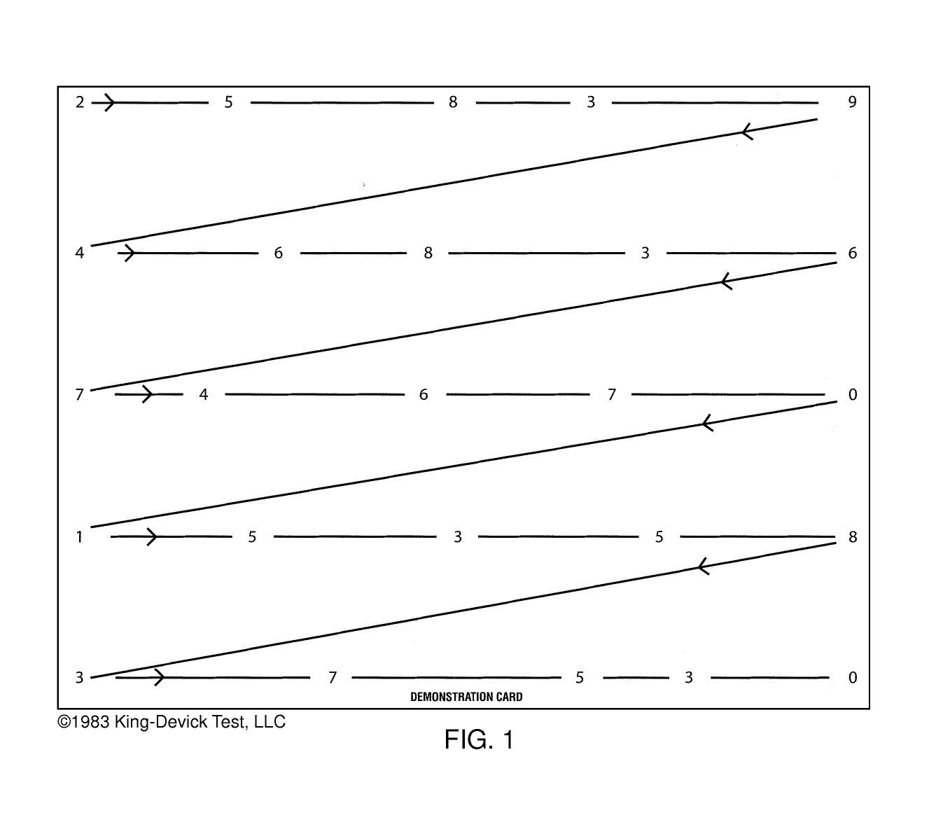

The K-D Test is administered by having a person read aloud numbers printed on a series of cards (demonstration page shown in FIG. 1) in a specified order and without moving the head, while a Test administrator times the Test and follows along on an answer key. The numbers are arranged on the card to force the person to use saccadic eye movements in order to read the numbers quickly and accurately. The Test administrator has a score card with the answer key (listing the correct sequence of numbers for each Test card) and a results table listing the maximum time and number of errors that persons of various ages can score and still have a “normal” Test result. A result is considered normal if the total time is below the maximum time and the total number of errors is below the maximum errors for an individual of the person's age.

Alternatively, the result may be compared to a particular person's baseline performance on the Test in order to determine whether the result is normal or abnormal. In this case, baseline measurements may be made for one or more persons, during, for example, routine medical examinations or at some other time. Those baseline results would then be made available by way of the person's medical records or other method of recordation for purposes of comparison if needed.

A person testing “abnormal” would then be treated as if the person has PD, or is at risk for developing PD. Such treatment could include recommending the person undergo additional testing for PD, referring the person to a neurologist or other specialist for additional testing for PD, recommending the person be administered a treatment for PD, or administering the person a treatment for PD, etc.

For patients who take the Test regularly, or frequently enough that they might learn the number patterns and thereby misleadingly improve their scores (perhaps even as their saccadic eye movements degrade), a battery of K-D Tests can be used, in which the spacing of the numbers is preserved but the numbers themselves are changed randomly or pseudo-randomly. Administering the Test on computer, such as on a suitably-sized tablet computer, can facilitate the use of a virtually limitless number variations.

I claim:

1. A method for treating Parkinson's disease (PD), comprising:
administering a King-Devick Test to a person suspected of having or developing PD;
determining whether the person has a “normal” or “abnormal” result for the King-Devick Test; and
administering a treatment for PD to the person if the result is “abnormal.”

2. The method of claim 1, wherein the result is determined by comparing the person's performance on the King-Devick Test to a prior baseline determined for the person.

3. The method of claim 1, wherein the result is determined by comparing the person's performance on the King-Devick Test to normative values.

4. The method of claim 1, wherein the method further comprises recommending the person undergo additional testing for PD before administering a treatment for PD to the person if the result is “abnormal.”

5. The method of claim 1, wherein the treatment is administered to the person substantially immediately after an “abnormal” result is obtained.

6. The method of claim 1, wherein the treatment is administered to the person before any other signs or symptoms of PD are observed.

7. The method of claim 6, wherein the treatment is administered substantially immediately after an “abnormal” result is obtained.

8. The method of claim 1, wherein the King-Devick Test is administered to the person daily to detect minute changes in the person's PD status.

9. The method of claim 8, wherein the King-Devick Test is administered on a tablet computer, and numbers used in the K-Devick Test are randomly- or pseudo-randomly generated, but the spacing of the numbers is preserved.

10. A method of screening for Parkinson's disease (PD), comprising:
administering a King-Devick Test to a person;
determining whether the person has a “normal” or “abnormal” result for the King-Devick Test; and
recommending the person undergo additional testing for PD if the result is “abnormal.”

11. The method of claim 10, wherein the result is determined by comparing the person's performance on the King-Devick Test to a prior baseline determined for the person.

12. The method of claim 10, wherein the result is determined by comparing the person's performance on the King-Devick Test to normative values.

13. The method of claim 10, wherein the recommending comprises referring the person to a specialist if the result is “abnormal.”

14. The method of claim 10, wherein the method further comprises recommending the person be administered a treatment for PD if the result is “abnormal.”

15. The method of claim 10, wherein the method further comprises administering a treatment to the person for PD if the result is “abnormal.”
